(19) United States
(12) Patent Application Publication
(10) Pub. No.: US 2022/0287939 A1
(43) Pub. Date: Sep. 15, 2022

(54) AGENT FOR MODIFYING PIGMENT TO DYE, AND COLORANT COMPOSITION COMPRISING THE DYE

(71) Applicant: AMOREPACIFIC CORPORATION, Seoul (KR)

(72) Inventors: Eunsil HAN, Yongin-si (KR); Minkyung SHIM, Yongin-si (KR)

(73) Assignee: AMOREPACIFIC CORPORATION, Seoul (KR)

(21) Appl. No.: 17/691,591

(22) Filed: Mar. 10, 2022

(30) Foreign Application Priority Data
Mar. 10, 2021 (KR) 10-2021-0031648

(51) Int. Cl. A61K 8/44; C09B 29/01; C09B 67/10; A61Q 1/02; A61K 8/36

(52) U.S. Cl. CPC A61K 8/445; C09B 29/0018; C09B 67/0015; A61Q 1/02; A61K 8/361; A61K 2800/43

(57) ABSTRACT

The present specification discloses a agent composition for modifying a pigment to dye that imparts tinting strength to the pigment, and a colorant composition in which a pigment is modified to dye using the same. According to the present disclosure, since the pigment has tinting strength on the skin, it is possible to realize various colors of the pigment as it is while having excellent durability when applied to the skin. Accordingly, the pigment may be comprised as a coloring agent in a cosmetic formulation requiring coloring by replacing the dye having a limitation in type and color when used in a cosmetic composition due to a stability problem for the skin. In addition, since the coloring agent according to the present disclosure has an irreversibly stabilized form, it can be widely used without being limited to the pH, formulation, etc. of the cosmetic composition.

### CROSS REFERENCE TO RELATED APPLICATION

The present application claims priority to Korean Patent Application No. 10-2021-0031648, filed on Mar. 10, 2021, the entire contents of which is incorporated herein for all purposes by this reference.

### BACKGROUND OF THE INVENTION

### Field of the Invention

The present specification describes a agent for modifying a pigment to dye, and a colorant composition comprising the modified pigment.

### Description of the Related Art

In general, cosmetic compositions include colorings to cover flaws in the skin or to impart a beautiful color to the skin. Colorings include dyes and pigments. Dye dissolves well in solvents and penetrates into the skin when applied to the skin, and the color is expressed by dyeing to show transparent and natural color development. However, there is a disadvantage in that the types of dyes that can be used in the cosmetic composition are extremely limited due to regulation due to the problem of stability on the skin. Accordingly, pigments have been mainly used as colorings in cosmetic compositions. Pigments are solid powders that are insoluble in water or organic solvents, and are opaque because they reflect a specific wavelength to show color. Pigments are largely divided into inorganic pigments and organic pigments. Inorganic pigments are mineral pigments and typically include titanium dioxide and iron oxide. Inorganic pigments are generally opaque and have excellent skin opacity, but when applied to the skin, flocculation appears and there is a possibility of skin damage. Organic pigments are provided in the form of a lake pigment and a toner pigment prepared in the form of a salt to improve stability, or a true pigment including a dye in which the raw material itself is insoluble. A lake pigment is a pigment made by adding an extender pigment and adsorbing a water-soluble dye to an insoluble substrate, and its usage is gradually decreasing due to low light fastness and low heat resistance. A toner pigment is a pigment insoluble prepared by adding precipitant such as metal salt to a water-soluble dye, like a lake pigment, but it is prepared by precipitation reaction through ion exchange without adding an extender pigment, unlike a lake pigment. A toner pigment is widely used because it has better color development and stability than a lake pigment. However, while the organic pigments can implement a variety of colors, there is a problem in that they do not penetrate into the skin because of low solubility in water or organic solvents, and thus the durability is deteriorated. Pigments have been developed to solve this problem, but most of them are the ones with improved dispersibility, and the pigments having tinting strength for skin have not yet been developed. Therefore, it is necessary to develop a coloring agent that is safe for skin while satisfying consumer needs for various colors and durability.

### SUMMARY OF THE INVENTION

In one aspect, an object to be achieved by the present disclosure is to provide a dyeing agent composition for a pigment that can be used to modify a pigment to a dye capable of implementing various colors to give the pigment a tinting strength on the skin.

In one aspect, an object to be achieved by the present disclosure is to provide a colorant composition that has excellent tinting strength and is safe for the skin when applied to the skin, and can implement various colors.

In one aspect, an object to be achieved by the present disclosure is to provide a method for manufacturing a colorant composition.

An embodiment of the present disclosure for achieving the above objects is a dyeing agent composition for a pigment, wherein the dyeing agent composition comprises a fatty acid and a neutralizing agent as active ingredients, wherein the pigment is a toner pigment, and wherein the fatty acid and the neutralizing agent undergo a saponification reaction with the pigment to solubilize the pigment.

An embodiment of the present disclosure provides a colorant composition comprising the pigment modified by the agent composition.

An embodiment of the present disclosure provides a method for preparing the colorant composition, which comprises the step of perform a saponification reaction by adding a neutralizing agent to a fatty acid and a toner pigment.

The present disclosure provides an agent composition for modifying a pigment to dye that imparts tinting strength to the pigment, and a colorant composition in which a pigment is modified using the same. According to the present disclosure, since the pigment has tinting strength on the skin, it is possible to realize various colors of the pigment as it is while having excellent durability when applied to the skin. Accordingly, the pigment may be comprised as a coloring agent in a cosmetic formulation requiring coloring by replacing the dye having a limitation in type and color when used in a cosmetic composition due to a stability problem for the skin. In addition, since the coloring agent according to the present disclosure has an irreversibly stabilized form, it can be widely used without being limited to the pH, formulation, etc. of the cosmetic composition.

### BRIEF DESCRIPTION OF THE DRAWINGS

FIG. 1 shows the tinting effect remaining on the skin in the photographs taken after the paper application once and twice, the photographs taken immediately after skin application, and the photographs taken after washed off with a tissue, water or soap immediately after application to the skin, in Example 1 (leftmost photo) of the present disclosure, and Comparative Examples 1 to 3, which are dyes commonly used in cosmetic compositions as comparative examples, in order from the left.

FIG. 2a shows the tinting effect remaining on the skin in the photographs taken immediately after application to the skin, the photographs taken after 10 minutes, and the photographs taken after washing, in Examples 1 to 3 of an embodiment of the present disclosure, in order from the left. In Comparative Example 3, the formulation was separated during manufacture, so that the tinting strength could not be evaluated.

FIG. 2b is a photograph taken while the formulation of Comparative Example 3 was separated.

FIG. 3 shows the tinting effect remaining on the skin in the photographs taken immediately after skin application, the photographs taken after tissue-off, and the photographs taken after water washing (washing) with water, in Examples 1, 4, 5, 6 to 8 of embodiments of the present disclosure, in order from the left. In Examples 4 and 7, the tinting strength could not be evaluated because the formulations were separated during manufacture.

FIG. 4 shows the tinting effect remaining on the skin in the photographs taken immediately after skin application, the photographs taken after tissue-off, and the photographs taken after water washing (washing), in Examples 6, 9 and 10 of embodiments of the present disclosure, in order from the left.

FIG. 5 shows the tinting effect remaining on the skin in the photographs taken immediately after skin application, the photographs taken after tissue-off, and the photographs taken after water washing (washing) in Example 1 of an embodiment of the present disclosure, and Comparative Examples 4 to 10, in order from the left.

FIG. 6 shows the tinting effect remaining on the skin in the photographs taken immediately after skin application, the photographs taken after 10 minutes, the photographs taken after tissue-off, the photographs taken after water washing (washing) in Examples 1, 2 and 11 of embodiments of the present disclosure, in order from the left.

FIG. 7 shows the tinting effect remaining on the skin in the photographs taken immediately after skin application, the photographs taken after 10 minutes, the photographs taken after tissue-off, the photographs taken after water washing (washing) in Examples 2, 12, 13 and 14 of embodiments of the present disclosure, in order from the left.

FIG. 8 shows the tinting effect remaining on the skin in the photographs taken immediately after skin application, the photographs taken after tissue-off, the photographs taken after water washing (wash-off), and the photographs taken after soap washing in the colorant composition of Example 1, and Examples 15, 16 and 17 comprising the colorant composition of Example 1 as a pigment base, in order from the left.

FIG. 9 shows photographs (top) of the appearances of the formulations after preparations of Example 18 (left) and Comparative Example 11 (right), and photographs (bottom) of the tinting degrees after skin application.

FIG. 10 shows photographs of the appearance taken after the preparations of Example 19 (left) and Comparative Example 12 (right).

FIG. 11 is a photograph of the appearance after the preparation of Comparative Example 13.

FIG. 12 is a photograph checking the tinting strength of Example 20. The left photograph shows the color immediately after application, and the right photograph shows the tinted color after application.

### DETAILED DESCRIPTION OF THE INVENTION

Hereinafter, embodiments of the present application will be described in more detail with reference to the accompanying drawings. However, the technology disclosed in the present application is not limited to the embodiments described herein and may be embodied in other forms. However, the embodiments introduced herein are provided so that the disclosed content may be thorough and complete, and the spirit of the present application may be sufficiently conveyed to those skilled in the art. In addition, those skilled in the art will be able to implement the spirit of the present application in various other forms within the scope that does not depart from the technical spirit of the present application.

An embodiment of the present disclosure provides an agent composition for modifying a pigment to a dye that comprises a fatty acid and a neutralizing agent as active ingredients, and in which the fatty acid and the neutralizing agent react with a toner pigment by saponification to solubilize the pigment.

An embodiment of the present disclosure provides a colorant composition comprising a pigment modified by the agent composition.

An embodiment of the present disclosure provides the use of a fatty acid and a neutralizing agent for use in the preparation of an agent composition for modifying a pigment to a dye.

As used herein, the term “pigment” refers to a general insoluble pigment, and refers to a kind of organic synthetic coloring agent having an opaque color and non-dyeing property.

As used herein, the term “dye” refers to an organic synthetic coloring agent having a transparent color and dyeability.

As used herein, the term “modified pigment” or “dyed pigment” is a saponification reactant of a pigment prepared by a saponification reaction of a pigment, a fatty acid, and a neutralizing agent, and exhibits properties similar to dyes by having a transparent color and dyeability. The modified pigment has hydrophilicity, so it is easy to disperse in a water type formulation, and there are no restrictions on international regulations compared to dyes, so it can be applied universally.

As an embodiment, the toner pigment is an insolubilized form of a dye, specifically, a water-soluble organic pigment to which a metal salt is bound. The toner pigment, which is a pigment to be dyed (modified to a dye) according to an embodiment of the present disclosure, may be used without limitation as long as it is a water-soluble organic pigment to which a salt is bound.

As an embodiment, the present disclosure can provide a new coloring agent that desalts a toner pigment by saponifying the toner pigment with an agent composition for modifying to dye comprising a fatty acid and a neutralizing agent, so that it can have tinting strength by penetrating into the skin when applied to the skin. Accordingly, it is possible to solve the limitation due to the stability problem of dyes used as conventional coloring agent, and to provide a coloring agent that can be implemented in various colors according to the types of toner pigments. In addition, since the present disclosure uses an amphoteric fatty acid and a neutralizing agent as a medium for acidification of the pigment to perform a saponification reaction rather than an ionization reaction, the dyed pigment has a stabilized backbone by a fatty acid having a long carbon chain structure and a neutralizing agent. The colorant composition comprising the dyed pigment according to the present disclosure has little change in the tinting strength according to pH and is irreversible, so it has excellent stability, and can be used as a colorant base in cosmetic compositions of various formulations.

As an embodiment, the fatty acid may be the fatty acid having 12 to 22 carbon atoms. More specifically, the fatty acid may comprise one or more selected from the group consisting of lauric acid, myristic acid, palmitic acid, stearic acid, and behenic acid. As an embodiment, depending on the type of fatty acid, there may be a difference in the formulation degree when comprised in the cosmetic composition. For example, stearic acid has excellent stability in a cream phase, and in the case of behenic acid and palmitic acid, the formulation may be separated during emulsification, so conditions may need to be adjusted. Lauryl acid and myristic acid have low viscosity, so they can be comprised in a high content in the composition, so it is convenient to use.

As an embodiment, the neutralizing agent is not limited as long as it is a basic material, and all of them can be used. As an embodiment, the basic material of the neutralizing agent may be a strong base or a weak base. Examples of the strong basic material comprise potassium hydroxide, sodium hydroxide, etc. The pH of the weak basic material may be 8 to 9. As an embodiment, the neutralizing agent may be an amine-based basic material. More specifically, the neutralizing agent may be an amine-based saturated aliphatic saturated alcohol. For example, the neutralizing agent may comprise one or more selected from the group consisting of amino methyl propanol, amino methyl propandiol, tromethamine, and triethanolamine.

As an embodiment, the weight ratio of the fatty acid and the neutralizing agent may vary depending on the type of the fatty acid and the neutralizing agent used. For example, the weight ratio of the fatty acid and the neutralizing agent comprised in the dyeing agent composition may be 1:0.1 to 4. More particularly, the weight ratio of the fatty acid and the neutralizing agent may be 0.1 or more, 0.2 or more, 0.3 or more, 0.4 or more, 0.5 or more, 0.6 or more, 0.7 or more, 0.8 or more, 0.9 or more, 1 or more, 1.1 or more, 1.2 or more, 1.3 or more, 1.4 or more, 1.5 or more, 1.6 or more, 1.7 or more, 1.8 or more, 1.9 or more, 2 or more, 2.1 or more, 2.2 or more, 2.3 or more, 2.4 or more, 2.5 or more, 2.6 or more, 2.7 or more, 2.8 or more, 2.9 or more, 3 or more, 3.1 or more, 3.2 or more, 3.3 or more, 3.4 or more, 3.5 or more, 3.6 or more, 3.7 or more, 3.8 or more or 3.9 or more, 1:4 or less, 3.9 or less, 3.8 or less, 3.7 or less, 3.6 or less, 3.5 or less, 3.4 or less, 3.3 or less, 3.2 or less, 3.1 or less, 3 or less, 2.9 or less, 2.8 or less, 2.7 or less, 2.6 or less, 2.5 or less, 2.4 or less, 2.3 or less, 2.2 or less, 2.1 or less, 2 or less, 1.9 or less, 1.8 or less, 1.7 or less, 1.6 or less, 1.5 or less, 1.4 or less, 1.3 or less, 1.2 or less, 1.1 or less, 1 or less, 0.9 or less, 0.8 or less, 0.7 or less, 0.6 or less, 0.5 or less, 0.4 or less, 0.3 or less or 0.2 or less. When the weight ratio is out of the range, the saponification reactivity may be low and the reaction efficiency may be reduced. When the unreacted fatty acid is comprised in the colorant composition because it comprises an excessive amount of fatty acid compared to the neutralizing agent, the tinting strength of the colorant composition may be reduced due to the fatty acid.

The modified pigment according to the embodiments of the present disclosure may have different tinting strength and stability depending on the weight ratio of the toner pigment, the fatty acid, and the neutralizing agent. In view of the above, the weight ratio of the toner pigment, the fatty acid, and the neutralizing agent according to an embodiment may be 0.1-1.5:5:0.5-20. In particular, an amount of the toner pigment may be 0.1 parts by weight or more, 0.2 parts by weight or more, 0.3 parts by weight or more, 0.4 parts by weight or more, 0.5 parts by weight or more, 0.6 parts by weight or more, 0.7 parts by weight or more, 0.8 parts by weight or more, 0.9 parts by weight or more, 1 parts by weight or more, 1.1 parts by weight or more, 1.2 parts by weight or more, 1.3 parts by weight or more or 1.4 parts by weight or more, and 1.5 parts by weight or less, 1.4 parts by weight or less, 1.3 parts by weight or less, 1.2 parts by weight or less, 1.1 parts by weight or less, 1 parts by weight or less, 0.9 parts by weight or less, 0.8 parts by weight or less, 0.7 parts by weight or less, 0.6 parts by weight or less, 0.5 parts by weight or less, 0.4 parts by weight or less, 0.3 parts by weight or less or 0.2 parts by weight or less, based on 5 parts by weight of the fatty acid. If the ratio is out of the above range, the reaction efficiency may be reduced, and thus the tinting strength may be insignificant or the increase in the tinting strength may not be large compared to the content of each ingredient used.

As one embodiment, the toner pigment may comprise one or more selected from the group consisting of a disodium salt of 4-(2-sulfo-p-tolylazo)-3-hydroxy-2-naphthoic acid, a calcium salt of 4-(2-sulfo-p-tolylazo)-3-hydroxy-2-naphthoic acid, and a calcium salt of 4-(1-sulfo-2-naphthylazo)-3-hydroxy-2-naphthoic acid. Specifically, the disodium salt of 4-(2-sulfo-p-tolylazo)-3-hydroxy-2-naphthoic acid may be an organic compound represented by the following Chemical Formula 1, and as an example, there is Red No. 201 (D&C Red no. 6, CI15850, Lithol Rubine B, Pigment Red 57). The calcium salt of 4-(2-sulfo-p-tolylazo)-3-hydroxy-2-naphthoic acid may be an organic compound represented by the following Chemical Formula 2, and as an example, there is Red No. 202 (D&C Red no. 7, CI15850:1, Lithol Rubine BCA, Pigment Red 57:1). The calcium salt of 4-(1-sulfo-2-naphthylazo)-3-hydroxy-2-naphthoic acid may be an organic compound represented by the following Chemical Formula 3, as an example, there is Red No. 220 (D&C Red no. 34, CI 15880:1, Deep Maroon, Pigment Red 63:1).

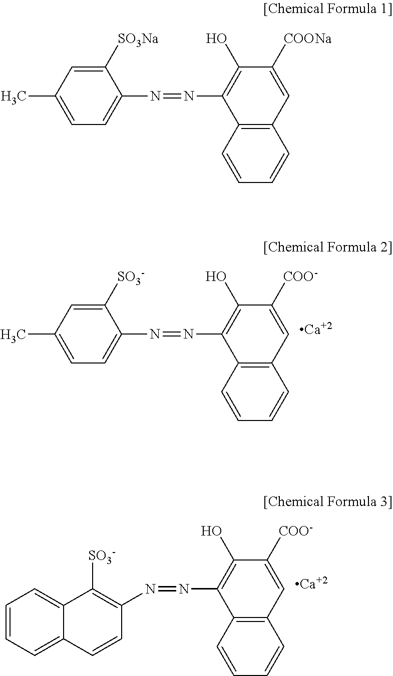

The pH drop due to the fatty acid is insignificant, and the pH of the colorant composition according to an embodiment may be 7 to 9.5. Particularly, the pH of the colorant composition according to an embodiment may be 7 or more, 7.1 or more, 7.2 or more, 7.3 or more, 7.4 or more, 7.5 or more, 7.6 or more, 7.7 or more, 7.8 or more, 7.9 or more, 8 or more, 8.1 or more, 8.2 or more, 8.3 or more, 8.4 or more, 8.5 or more, 8.6 or more, 8.7 or more, 8.8 or more, 8.9 or more, 9 or more, 9.1 or more, 9.2 or more, 9.3 or more, or 9.4 or more, and 9.5 or less, 9.4 or less, 9.3 or less, 9.2 or less, 9.1 or less, 9 or less, 8.9 or less, 8.8 or less, 8.7 or less, 8.6 or less, 8.5 or less, 8.4 or less, 8.3 or less, 8.2 or less, 8.1 or less, 8 or less, 7.9 or less, 7.8 or less, 7.7 or less, 7.6 or less, 7.5 or less, 7.4 or less, 7.3 or less, 7.2 or less or 7.1 or less. As described above, since the colorant composition according to the present disclosure has neutral or weak basic properties, it can be used without limitation in various formulations of the cosmetic composition.

An embodiment of the present disclosure may provide a cosmetic composition comprising the above-described colorant composition.

In an embodiment, the cosmetic composition according to the present disclosure may be formulated by comprising a cosmetically or dermatologically acceptable medium or base. These are all formulations suitable for topical application, and they may be provided, for example, in the form of solutions, gels, solids, kneaded dry products, emulsions obtained by dispersing an oily phase in the form of a water phase, suspensions, microemulsions, microcapsules, microgranules or ionic (liposomes) and nonionic vesicular dispersant, or in the form of a cream, skin, lotion, powder, ointment, spray or concealer stick. As an embodiment, the cosmetic composition may be a color cosmetic composition, and more specifically, may be a cosmetic composition for lips, but is not limited thereto and may be used as a pigment base in a cosmetic composition of various formulations. As an embodiment, the lip cosmetic composition may comprise a cosmetic composition for lipstick, lip gloss, lip balm, tint, lip pencil, etc. These compositions can be prepared according to conventional methods in the art. As an embodiment, the cosmetic composition according to the present disclosure may comprise, together with the active ingredient, preferably other ingredients capable of giving a synergistic effect to the main effect, within a range that does not impair the main effect. Also, in the cosmetic composition, other ingredients other than the active ingredient of the present disclosure may be appropriately selected and formulated by those skilled in the art without difficulty depending on the formulation or purpose of use of the other cosmetic composition. In addition, as an embodiment, the cosmetic composition of the present disclosure may comprise other ingredients that are usually formulated in the cosmetic composition, if necessary, in addition to the above ingredients. For example, there are organic pigments, inorganic pigments, dyes, moisturizers, emollients, organic powders, UV absorbers, preservatives, disinfectants, antioxidants, plant extracts, pH adjusters, alcohols, fragrances, blood circulation promoters, cooling agents, antiperspirant agents, purified water, and the like. Other compounding ingredients that may be comprised in the cosmetic composition of the present disclosure are not limited thereto, and the compounding amount of the ingredients is possible within a range that does not impair the purpose and effect of the present disclosure. As an embodiment, the colorant composition may be comprised in an amount of 0.1 to 20% by weight based on the total weight of the cosmetic composition. Particularly, the colorant composition may be comprised, based on the total weight of the cosmetic composition, in an amount of 0.1% by weight or more, 0.5% by weight or more, 1% by weight or more, 1% by weight or more, 2% by weight or more, 3% by weight or more, 4% by weight or more, 5% by weight or more, or 10% by weight or more, and 20% by weight or less, 15% by weight or less, 10% by weight or less, 5% by weight or less, 2% by weight or less, 1% by weight or less or 0.5% by weight or less. More specifically, the colorant composition may be comprised in an amount of 0.5 to 2% by weight based on the total weight of the cosmetic composition. When the colorant composition is comprised in an amount of less than 0.5% by weight, the tinting strength may be insignificant, and when the colorant composition exceeds 2% by weight, the increase in tinting strength may not be large compared to the amount of the comprised colorant composition.

In the present disclosure, the cosmetic composition comprising the solubilized pigment may further comprise functional additives and/or ingredients comprised in a general cosmetic composition. Examples of the functional additives comprise water-soluble vitamins, oil-soluble vitamins, high-molecular peptides, high-molecular polysaccharides, sphingolipids, seaweed extracts, and the like. The ingredients comprised in the general cosmetic composition are oils and fats, moisturizers, emollients, surfactants, organic powders, UV absorbers, preservatives, bactericides, antioxidants, plant extracts, pH adjusters, alcohols, pigments, fragrances, blood circulation promoters, cooling agents, antiperspirant agents, purified water, etc.

An embodiment of the present disclosure may provide a method for modifying the above-described pigment to dye, or a method for manufacturing the colorant composition.

As an embodiment, the method may comprise the step of performing a saponification reaction by adding a neutralizing agent to a fatty acid and a toner pigment.

Specifically, the saponification reaction may comprise the steps of dispersing the toner pigment in an oil phase in which the fatty acid is dissolved; and adding a water phase comprising the neutralizing agent to the oil phase to perform the saponification reaction. As an embodiment, the temperature of the oil phase, the water phase, or the oil phase part and the water phase may be 50 to 70° C., but the temperature may vary depending on the temperature at which the fatty acid is dissolved in the oil phase.

As an embodiment, the method may further comprise the step of cooling to 20 to 40° C., after the saponification reaction. As an embodiment, the manufacturing method may further comprise a degassing step.

Hereinafter, the configuration and effect of the present disclosure will be described in more detail with reference to embodiments and drawings. However, the following examples and drawings are provided for the purpose of illustration only to help the understanding of the present disclosure, and the scope and scope of the present disclosure are not limited thereto.

### Test Example 1

As an embodiment of the present disclosure, a colorant composition comprising a dyed pigment (a modified pigment to dye) was prepared according to the following method, and the effects related to its tinting strength were compared with commercially available dyes.

Specifically, in the ingredients shown in Table 1 below, an oil phase was prepared by adding stearic acid, which is the fatty acid, to octyldodecanol and polyglyceryl-2 triisostearate, which are the oils, heating them to 65° C. for dissolving, and then adding and dispersing the toner pigment (R #6 Na salt) in the oil. A water phase was prepared by dissolving aminomethyl propanediol, which is the neutralizing agent, in deionized water while heating it to 65° C. The water phase was put into the oil phase, and the composition was sufficiently saponified for 20 minutes based on 800 g of the composition, then cooled to 30° C., and degassed to prepare a colorant composition (Example 1). The content or each ingredient means % by weight based on the total weight of the finally prepared colorant composition.

TABLE 1

| | content |
|---|---|
| | (% by |
| Ingredient name | weight) |
| octyldodecanol(Ingredient name: Eutanol G, | 25 |
| Manufacturer: BASF) | |
| polyglyceryl-2 triisostearate (Ingredient name: | 3.5 |
| Cosmol 43SV, Manufacturer: The Nisshin OilliO, Ltd.) | |
| stearic acid(Ingredient name: Triple pressed stearic | 5 |
| acid/Palmera 1802, Manufacturer: PALM-OLEO SDN. BHD. ) | |
| toner pigment(R#6 Na salt(CI 15850); Ingredient name: | 1.5 |
| D&C Red No. 6 Sodium Salt C19-6619, Manufacturer: | |
| sumchemical) | |
| aminomethyl propanediol (Ingredient name: AMPD; | |
| Manufacturer: ANGUS CHEMICAL COMPANY) | |
| D.I. water | To 100 |
| Total | 100 |

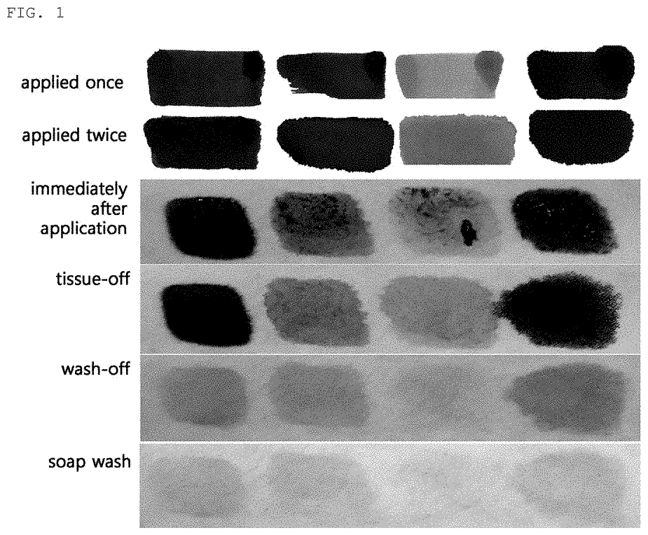

This was compared with Comparative Example 1(Red No. 40 07700-C, sensient), Comparative Example 2 (Carmin Soluble W 3014, sensient) and Comparative Example 3 (TPP Red No. 227, KISHI KASEI CO., LTD.), which are dyes used in the cosmetic field, and the results were shown in FIG. 1 and Table 2 below.

In this case, a paper application was performed by applying Example 1, Comparative Example 1, 2 or 3 once on white paper with a tip applicator, and then applying (reapplying) three additional times.

The skin application was performed by applying Example 1, Comparative Example 1, 2 or 3 to the arm five times with the tip applicator.

A tissue-off was carried out by applying Example 1, Comparative Example 1, 2 or 3 to the arm five times with the tip applicator, and after 5 minutes, placing a tissue on the application and pressing firmly to remove moisture.

A wash-off (water washing) was carried out by applying Example 1, Comparative Example 1, 2 or 3 to the arm five times with the tip applicator, and washing the applied area with running water after 5 minutes by a hand.

A soap wash was carried out by applying Example 1, Comparative Example 1, 2, or 3 to the arm five times with the tip applicator, and after 5 minutes, applying a liquid soap (Product name: Happy Bath Bubble Hand Wash, Manufacturer: Amore Pacific) to the area under running water.

After the paper application one time and two times, immediately after the skin application, after tissue-off, after wash-off, or after soap wash, pictures of the paper or skin were taken and shown in FIG. 1. Based on the photographs of FIG. 1, the tinting strength, tinting retention degree, darkening generation, appearance, and uniformity of the application of each of Example 1 and Comparative Examples 1 to 3 were evaluated in 5 stages, and were shown in Table 2 below. When the number of stars (★) is 5, it means that each performance is excellent. ⋆ means lower performance than ★.

TABLE 2

| | | Comparative | Comparative | Comparative |
|---|---|---|---|---|
| | Example 1 | Example 1 | Example 2 | Example 3 |
| tinting | ★★★★ | ★★★ | ⋆ | ★★★★★ |
| strength | | | | |
| tinting | ★★★★★ | ★★★ | ⋆ | ★★★★★ |
| retention | | | | |
| degree | | | | |
| darkening | ★★★★★ | ★★★ | ★★★★★ | ★★★ |
| appearance | ★★★★★ | ⋆ | ⋆ | ★ |
| uniformity | ★★★★★ | ★ | ★ | ★ |

As a result, in a case of the paper application, Example 1 and Comparative Example 1 showed a warm tone, and Comparative Examples 2 and 3 showed a cool tone. This means that the present disclosure can provide a warm-toned colorant composition by replacing the dye of Comparative Example 1, which has a low tinting strength and has limited use due to discoloration. The color intensity of Example 1 is significantly higher than that of Comparative Example 2 and more distinct than Comparative Example 1, which shows a similar warm-tone color, as a result of paper application or skin application in FIG. 1. Thus, it can be seen that the colorant composition prepared by dyeing the pigment according to the present disclosure can implement a vivid color. In addition, as shown in FIG. 1, in Comparative Examples 1 to 3, which are conventional dyes, stains occurred due to water condensing due to the surface tension of water when applied to the skin, resulting in poor uniformity. However, the colorant composition according to the present disclosure was uniformly tinted due to excellent skin affinity due to the saponification reaction. In the case of Comparative Examples 1 and 3, the tissue-off results showed that the darkening part appeared outside the application range, i.e., the darkening. However, Example 1 of the present disclosure was uniformly tinted without darkening, and was found that the tinting retention degree was excellent even after wash-off and soap wash.

Table 3 below shows the results of checking the formulation stability of the colorant composition according to an embodiment of the present disclosure. Specifically, three samples were prepared with the same composition and method as in Example 1 and stored for 1 month in a low to high temperature cycle, and the change in the content of the dyed pigment was repeatedly checked. The low to high temperature cycle is a 24-hour cycle in the temperature range of −10° C. to 45° C. That is, the temperature cycle is performed by maintaining at −10° C. for 7 hours, then maintaining at 30° C. for 7 hours after the temperature rise for 1 hour, then maintaining at 45° C. for 7 hours after the temperature rise for 1 hour, then maintaining at −10° C. for 7 hours after the temperature drop for 1 hour. This temperature cycle was repeated for 1 month.

TABLE 3

Content of dyed pigment
(% by weight based on the
total weight of Example 1)

| | Date of analysis: | Date of analysis: |
|---|---|---|
| Example 1 | 2020 Nov. 3 | 2020 Dec. 3 |
| Sample 1 (date of | 1.03 | 1.57 |
| manufacture: | | |
| 2020 Aug. 28) | | |
| Sample 2(date of | 1.28 | 1.71 |
| manufacture: | | |
| 2020 Oct. 7) | | |
| Sample 3(date of | 1.13 | 1.57 |
| manufacture: | | |
| 2020 Nov. 2) | | |

As a result, it can be seen that the degree of dyeing reaction increased over time in all three samples after preparing the colorant composition based on the initial content of the pigment of 1.5% by weight, and did not return irreversibly.

### Test Example 2

In order to compare the tinting strength and the formulation stability according to the weight ratio of the fatty acid and the neutralizing agent according to an embodiment of the present disclosure, the following experiment was performed.

The colorant composition used in the test was prepared in the same manner as in Example 1 of Test Example 1, but the contents (% by weight) of the fatty acid, pigment and neutralizing agent were adjusted as shown in the following table. Then, in the same manner as in Test Example 1, the tinting strength and the formulation stability were compared immediately after the skin application, after 10 minutes, and after water washing (washing).

TABLE 4

| | Example | Example | Example |
|---|---|---|---|
| Ingredient name | 1 | 2 | 3 |
| stearic acid (Ingredient name: | 5.0 | 5.0 | 5.0 |
| Triple pressed stearic | | | |
| acid/Palmera 1802, Manufacturer: | | | |
| PALM-OLEO SDN. BHD.) | | | |
| toner pigment(R#6 Na salt(CI | 1.5 | 1.5 | 1.5 |
| 15850); Ingredient name: D&C Red | | | |
| No. 6 Sodium Salt C19-6619, | | | |
| Manufacturer: sumchemical) | | | |
| aminomethyl propanediol | 1.0 | 0.5 | 0.2 |
| (Ingredient name: AMPD; | | | |
| Manufacturer: ANGUS CHEMICAL | | | |
| COMPANY) | | | |
| pH | 9.03 | 8.68 | 7.66 |

(% by weight)

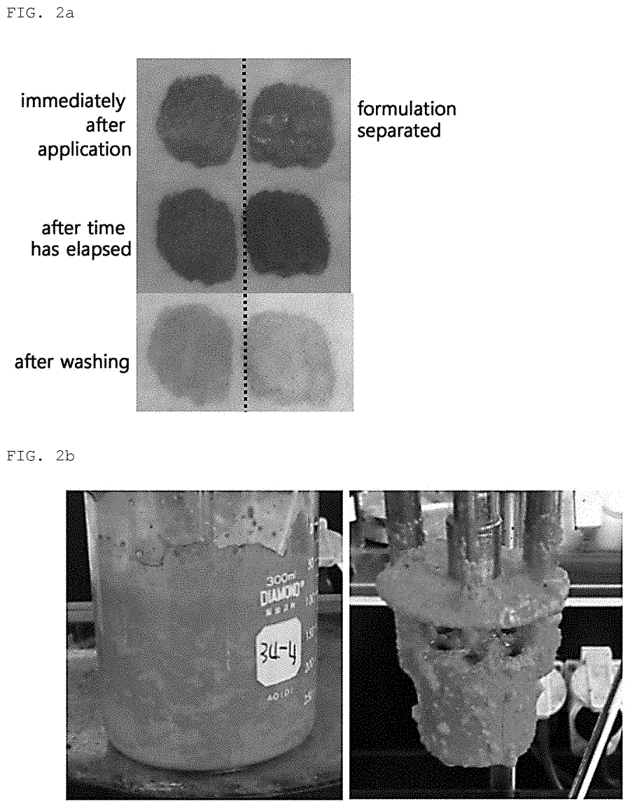

As a result, in Examples 1 and 2, stable formulations were formed, and as shown in FIG. 2a, it was found that the tinting strength was excellent. On the other hand, in the case of Example 3, the formulation was separated during manufacture, and thus the tinting strength could not be evaluated (see FIGS. 2a and 2b). This means that as the content of the fatty acid to the neutralizing agent increases, as shown in the table above, the fatty acid affects the formulation stability and the tinting strength rather than affecting the pH.

### Test Example 3

In order to compare the tinting strength according to the type of fatty acid according to an embodiment of the present disclosure, the following experiment was performed.

A colorant composition was prepared in the same manner as in Example 1 of Test Example 1, but the contents of the fatty acid and the neutralizing agent were adjusted as follows, and the tinting strength and the formulation stability were compared in the same manner as in Test Example 1. In this case, the degree of neutralization was 50.

TABLE 5

| Ingredient | Example | Example | Example | Example | Example | Example |
|---|---|---|---|---|---|---|
| name | 1 | 4 | 5 | 6 | 7 | 8 |
| stearic acid | 5.0 | — | — | — | — | — |
| behenic acid | — | 5.0 | — | — | — | — |
| isostearic | — | — | 5.0 | — | — | — |
| acid | | | | | | |
| myristic | — | — | — | 5.0 | — | — |
| acid | | | | | | |
| palmitic | — | — | — | — | 5.0 | — |
| acid | | | | | | |
| lauryl acid | — | — | — | — | — | 5.0 |
| neutralizing | 1.0 | 0.7 | 1.0 | 1.1 | 0.4 | 0.8 |
| agent (amino | | | | | | |
| methyl | | | | | | |
| propanediol) | | | | | | |
| pH | 9.03 | — | 9.36 | 8.96 | — | 8.11 |

(% by weight)

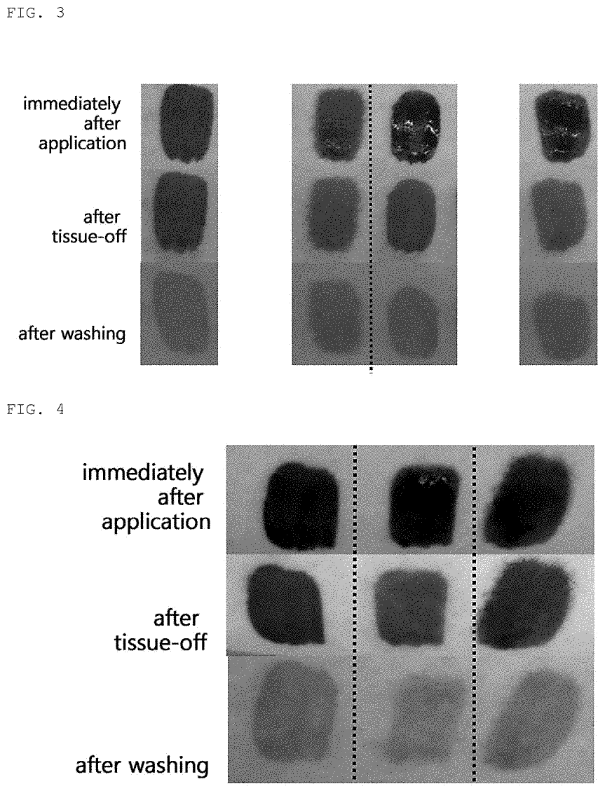

As a result, as shown in Table 5 and FIG. 3, there was no significant difference in the tinting strength depending on the type of fatty acid, but there was a difference in the formulation. Examples 1 and 5 in which stearic acid or isostearic acid was comprised formed stable cream formulations. In Examples 4 and 7 in which behenic acid or palmitic acid was comprised, the formulation was separated during manufacture, and pH or tinting strength could not be checked. Examples 6 and 8 in which lauric acid or myristic acid was comprised were found to be of low viscosity, convenient for use in various formulations of the composition.

### Test Example 4

The following experiment was conducted to compare the tinting strength, the safety (pH) and the formulation stability according to the weight ratio of the fatty acid and the neutralizing agent according to an embodiment of the present disclosure.

A colorant composition was prepared in the same manner as in Example 1 of Test Example 1, but the types and contents of the fatty acid and the neutralizing agent were adjusted as follows, and the tinting strength and the formulation stability were compared in the same manner as in Test Example 1.

TABLE 6

| Ingredient name | Example 6 | Example 9 | Example 10 |
|---|---|---|---|
| myristic acid | 5.0 | 5.0 | 5.0 |
| neutralizing agent (amino | 1.1 | 1.7 | 0.6 |
| methyl propanediol) | | | |
| degree of neutralization | 50 | 75 | 25 |
| (%) | | | |
| pH | 8.96 | top: 9.19 | 8.54 |
| | | bottom: | |
| | | 9.00 | |

(% by weight)

As a result, as shown in FIG. 4, in the case of Example 9 with a degree of neutralization of 751, the formulation was separated and a layer structure appeared, and in Example 10 with a degree of neutralization of 25%, the tinting retention degree was rather increased. It seems that the fatty acid acts as a wax in a structure and the tinting retention degree is increased.

### Test Example 5

The following experiment was conducted to compare the tinting strength, the safety (pH) and the formulation stability when an acid other than the fatty acid was further comprised in the dyeing reaction of the pigment according to an embodiment of the present disclosure.

A colorant composition was prepared in the same manner as in Example 1 of Test Example 1, but the types and contents of the pigment, the fatty acid and the neutralizing agent were adjusted as follows, and the tinting strength was compared in the same manner as in Test Example 1. The formulation stability was evaluated by observing the degree of separation of the formulation for 4 weeks at room temperature after storage for 3 days in the same temperature cycle as in Test Example 1. It was evaluated as o when the formulation of the composition remained stable until 4 weeks had elapsed, it was evaluated as a when the formulation was partially separated but maintained again by shaking during the 4 week period, and it was evaluated as X when the formulation was separated and a pigment band was formed or some ingredients subsided.

TABLE 7

| | | Compar- | Compar- | Compar- | Compar- | Compar- | Compar- | Compar- |
|---|---|---|---|---|---|---|---|---|
| | | ative | ative | ative | ative | ative | ative | ative |
| Ingredient | Example | Example | Example | Example | Example | Example | Example | Example |
| name | 1 | 4 | 5 | 6 | 7 | 8 | 9 | 10 |
| stearic acid | 5.0 | 5.0 | 5.0 | 5.0 | 5.0 | 5.0 | 5.0 | 5.0 |
| lactic acid | — | 0.2 | 0.2 | 0.2 | 0.4 | 0.6 | — | — |
| citric acid | — | — | — | — | — | — | 0.2 | 0.6 |
| neutralizing | 1.0 | 1.0 | 1.0 | 1.0 | 1.0 | 1.0 | 1.0 | 0.2 |
| agent (amino | | | | | | | | |
| methyl | | | | | | | | |
| propanediol) | | | | | | | | |
| pH | 9.03 | 8.76 | 8.81 | 8.7 | 8.41 | 8.19 | 8.65 | 4.97 |
| Stability | ◯ | X | X | X | X | X | Δ | X |

(% by weight)

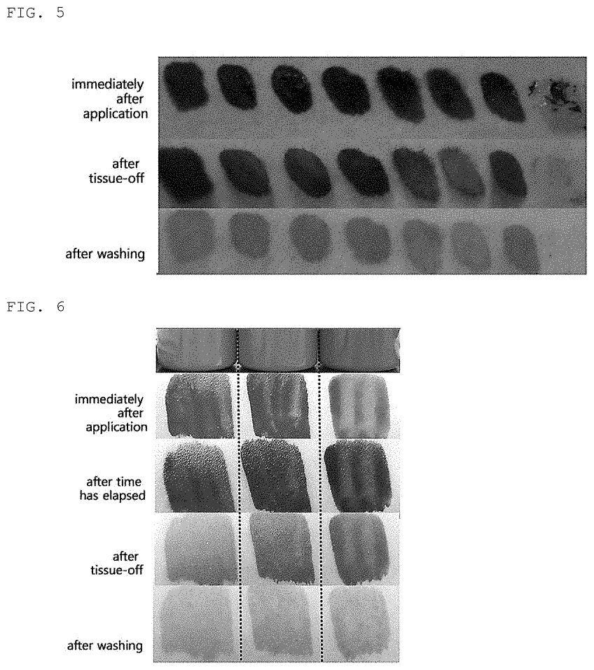

As a result, when a small amount of an acid other than the fatty acid such as citric acid or lactic acid was further comprised in the composition of the present disclosure, the formulation was separated and the stability was lowered, which was determined because the acid was not mixed with the saponification reaction product. In addition, as shown in FIG. 5, as the content of the acid was increased, the tinting strength was further decreased, and in particular, in Comparative Example 10, almost no tinting was achieved.

### Test Example 6

In order to compare the tinting strength and the tinting color according to the type of the pigment in the colorant composition according to an embodiment of the present disclosure, the following experiment was conducted.

The tinting strengths of Examples 1, 2, and 11 (R #7) were compared in the same manner as in Test Example 1. In this case, Example 11 was prepared in the same manner as in Example 1, but Red No. 202 (C19-7711 Soft-Tex® D&C Red No. 7 Calcium Lake, SUN CHEMICAL) was used as the pigment.

As a result, as shown in FIG. 6, there were differences in the appearance of each composition and the color immediately after the application, and the tinting strength and the tinting retention degree were the same. This is because the difference between Examples 1, 2 and 11 was only in the type of salt bound to the pigment used, but the salt bound was desalted due to the saponification reaction.

### Test Example 7

In order to check whether the tinting color of the colorant composition according to an embodiment of the present disclosure can be diversified, the coloring agents in the table below were further added to the colorant composition of Example 2, and their respective colors and tinting strength were checked in the same manner as in Test Example Since the coloring agents of blue color and pinky red color in the table below are dissolved directly in water, the coloring agents were additionally added after the preparation of the composition of Example 2.

TABLE 8

| | Example | Example | Example | Example |
|---|---|---|---|---|
| Ingredient name | 2 | 12 | 13 | 14 |
| stearic acid | 5.00 | 5.00 | 5.00 | 5.00 |
| toner pigment(D&C | 1.5 | 1.5 | 1.5 | 1.5 |
| RED NO. 6 SODIUM | | | | |
| SALT C19-6619, | | | | |
| SUN CHEMICAL) | | | | |
| blue | — | 0.01 | — | — |
| colorant(Ingredient | | | | |
| name: TPP BLUE | | | | |
| NO. 1, | | | | |
| Manufacturer: | | | | |
| KISHI KASEI | | | | |
| CO., LTD.) | | | | |
| pinky read | — | — | 0.10 | 0.20 |
| colorant(Ingredient | | | | |
| name: Unicert | | | | |
| Red K7054-J, | | | | |
| Manufacturer: | | | | |
| sensient) | | | | |
| neutralizing | 1.00 | 1.00 | 1.00 | 1.00 |
| agent (amino | | | | |
| methyl | | | | |
| propanediol) | | | | |

(% by weight)

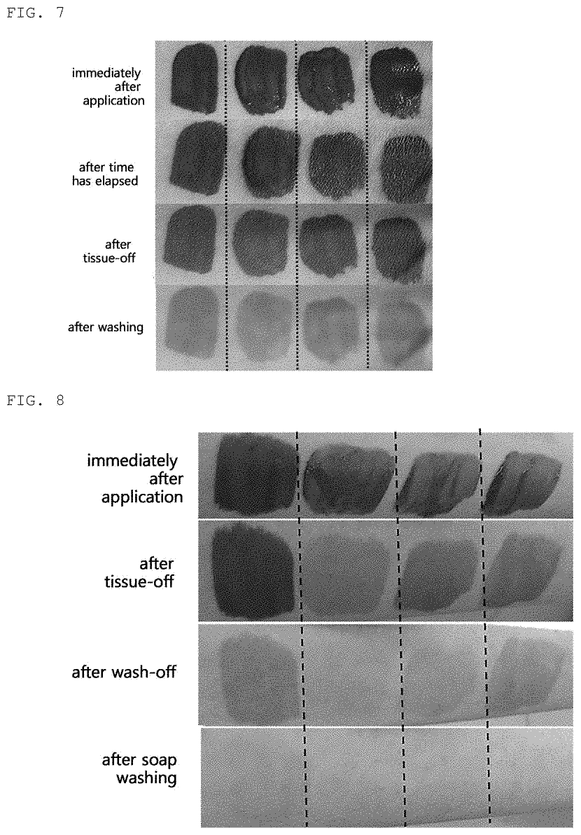

As a result, it can be checked that coloring agents of various colors can be prepared as shown in FIG. 7.

### Test Example 8

In order to check the possibility of using the colorant composition according to an embodiment of the present disclosure as a pigment base, the colorant composition was added as a pigment when preparing the composition of various emulsion formulations. Compositions of oil-in-water, water-in-oil and water-in-silicone oil were prepared according to the following methods by a conventional manufacturing method in the art. In this case, the content of each ingredient was described in Tables 9 to 11, and Example 1 of Test Example 1 was added as a colorant.

TABLE 9
Example 15: oil-in-water type composition

| | | Content |
|---|---|---|
| | Ingredient name | (% by weight) |
| 1 | 2-octyldodecanol | 10 |
| 2 | diisostearyl malate | 8 |
| 3 | dipnenylsiloxy phenyl trimethicone | 5 |
| 4 | bis-diglyceryl polyacyladipate-2 | 6 |
| 5 | steareth-21 | 3 |
| 6 | steareth-2 | 1 |
| 7 | cetearyl alcohol | 2 |
| 8 | D.I. water | To 100 |
| 9 | glycerin | 3 |
| 10 | butylene glycol | 5 |
| 11 | disodium EDTA | 0.05 |
| 12 | trehalose | 1 |
| 13 | ethanol | 1 |
| 14 | 1,2-hexanediol | 1 |
| 15 | pentylene glycol | 1 |
| 16 | glyceryl caprylate | 0.1 |
| 17 | colorant composition of Example 1 | 20 |
| 18 | hydroxyethyl acrylate/sodium | 1.5 |
| | acryloyldimethyl taurate copolymer | |

The oil-in-water type composition of Example 15 was prepared by the following preparation method.

1) a step of adding the ingredients 1 to 7 of Table 9 in a container and mixing them after heating to 65° C.;

2) a step of adding the ingredients 8 to 16 of Table 9 in a separate container and mixing them after heating to 65° C.;

3) a step of adding and dispersing the ingredient 17 of the colorant composition of the present disclosure in the container in which the ingredients 8 to 16 are mixed;

4) a step of adding the mixture of the ingredients 1 to 7 in the container in which the colorant composition is dispersed, for emulsifying;

5) a step of adding a thicker of the ingredient 11 for thickening, after the emulsifying; and

6) a step of cooling the resultant of step 5) to 30° C.

TABLE 10
Example 16: water-in-oil type composition

| | | Content (% |
|---|---|---|
| | Ingredient name | by weight) |
| 1 | octyldodecanol | 18.9 |
| 2 | ethylcellulose | 2.1 |
| 3 | trimethylsiloxyphenyl dimethicone | 8.5 |
| 4 | sorbitan stearate | 6 |
| 5 | polysorbate 60 | 1.5 |
| 6 | D.I. water | To 100 |
| 7 | glycerine | 5 |
| 8 | ethylhexylglycerin | 0.1 |
| 9 | phenoxyethanol | 0.4 |
| 10 | colorant composition of Example 1 | 20 |
| 11 | polyacrylate- | 0.6 |
| | 13*polyisobutene*water/aqua/eau*polysorbate | |
| | 20*sorbitan isostearate | |
| 12 | ethanol | 3 |

The water-in-oil type composition of Example 16 was prepared by the following preparation method.

1) a step of adding the ingredients 1 to 5 of Table 10 in a container and mixing them after heating to 65° C.;

2) a step of adding the ingredients 6 to 10 of Table 10 in a separate container and mixing them after heating to 65° C.;

3) a step of adding the mixture of the ingredients 1 to 5 in the container in which the ingredients 6 to 10 are mixed for emulsifying;

4) a step of adding a thicker of the ingredient 18 for thickening, after the emulsifying, and adding and mixing the ingredient 12; and

5) a step of cooling the resultant of step 5) to 30° C.

TABLE 11
Example 17: water-in-silicone oil type composition

| | | Content (% |
|---|---|---|
| | Ingredient name | by weight) |
| 1 | D.I. water | 15 |
| 2 | glyceryl polymethacrylate | 3 |
| 3 | pentylene glycol | 1 |
| 4 | 1,2-hexanediol | 1 |
| 5 | disodium EDTA | 0.05 |
| 6 | colorant composition of Example 1 | 20 |
| 7 | cetyl PEG/PPG-10/1 dimethicone | 1.5 |
| 8 | lauryl PEG-9 polydimethylsiloxyethyl dimethicone | 1.5 |
| 9 | lauryl polyglyceryl-3 polydimethylsiloxyethyl | 1 |
| | dimethicone | |
| 10 | caprylyl methicone | |
| 11 | bis-diglyceryl polyacyladipate-2 | 2.5 |
| 12 | dimethicone*trisiloxane | To 100 |
| 13 | trimethylsiloxysilicate | 5 |
| 14 | hydrogenated polyisobutene | 2 |
| 15 | dimethicone*dimethicone/vinyl dimethicone | 6.2 |
| | crosspolymer | |
| 16 | cyclopentasiloxane*dimethicone crosspolymer | 6 |
| 17 | dimethicone*dimethicone crosspolymer | 18 |
| 18 | aluminum starch octenylsuccinate | 1 |
| 19 | vinyl dimethicone/methicone silsesquioxane | 1.3 |
| | crosspolymer | |

The water-in-silicone oil type composition of Example 17 was prepared by the following preparation method.

1) a step of adding the ingredients 1 to 5 of Table 11 in a container and mixing them after heating to 65° C.;

2) a step of adding and mixing the ingredient 6 of the colorant composition of the present disclosure in the container in which the ingredients 1 to 5 are mixed;

3) a step of adding the ingredients 7 to 19 of Table 11 in a separate container and mixing them after heating to 65° C.;

4) a step of adding the mixture comprising the colorant composition to the mixture comprising the ingredients 7 to 19 for emulsifying;

5) a step of cooling the resultant of step 4) to 30° C.

As a result, the formulations of all three compositions were stably prepared. In addition, the tinting strength of each composition was evaluated in the same manner as in Test Example 1, and is shown in FIG. 8. FIG. 8 shows the colorant composition of Example 1 and Examples 15, 16 and 17 comprising the colorant composition of Example 1 as a pigment base, sequentially from the left. As a result, it can be confirmed that when the colorant composition according to an embodiment of the present disclosure is comprised as a pigment base in the compositions of various formulations, the tinting effect is exhibited while having a stable formulation.

### Test Example 9

A colorant composition was prepared by dyeing a toner pigment using the dyeing agent composition according to an embodiment of the present disclosure. Then, the tinting effects and the formulation stabilities when the colorant composition was added as a pigment base of the cosmetic composition, and when the fatty acid and the neutralizing agent were simply mixed together with the toner pigment in the manufacturing step of the cosmetic composition were compared and evaluated.

TABLE 12
Water-in-oil type emulsified formulation

| | | Example | Example |
|---|---|---|---|
| | Ingredient name | 18 | 11 |
| 1 | phenyl trimethicone | 0.96 | 0.96 |
| 2 | trimethylsiloxysilicate*poly- | 26.4 | 26.4 |
| | propylsilsesquioxane | | |
| 3 | cyclopentasiloxane*dimethiconol | 2.56 | 2.56 |
| 4 | behenyl alcohol | 0.384 | 0.384 |
| 5 | diisostearyl malate | 1.6 | 1.6 |
| 6 | polysorbate 60 | 0.7 | 0.7 |
| 7 | PEG-10 dimethicone | 3 | 3 |
| 8 | stearic acid | — | 1 |
| 9 | octyldodecanol | — | 3 |
| 10 | polyglyceryl-2 triisostearate | — | 0.7 |
| 11 | toner pigment(R#6 Na salt(CI 15850)) | — | 0.3 |
| 12 | D.I water | to 100 | to 100 |
| 13 | aminomethyl propandiol | — | 0.2 |
| 14 | glycerine | 0.75 | 0.75 |
| 15 | colorant composition of Example 1 | 20 | — |
| 16 | 1,2-hexanediol | 1.5 | 1.5 |
| 17 | pentylene glycol | 1 | 1 |
| 18 | ammonium acryloyl dimethyltaurate/VP | 0.5 | 0.5 |
| | copolymer | | |

(% by weight)

The water-in-oil type composition of Example 18 was prepared by the following preparation method.

1) a step of adding the ingredients 1 to 7 of Table 12 in a container and dispersing them after dissolving at 65° C.;

2) a step of adding the ingredients 12, 14 to 17 of Table 12 in a container, dispersing them, and then heating to 65° C.;

3) a step of adding the resultant of step 2) to the resultant of step 1) for emulsifying;

4) a step of adding the ingredient 18 of Table 12 to the resultant of step 3) for thickening; and

5) a step of cooling the result of step 4) to 30° C.

The water-in-oil type composition of Comparative Example 11 was prepared by the following preparation method.

1) a step of adding the ingredients 1 to 11 of Table 12 in a container and dissolving them at 65° C., and then dispersing the ingredient 11;

2) a step of adding the ingredients 12 to 14, 16 and 17 of Table 12 in a container, dispersing them, and heating to 65° C.;

3) a step of adding the resultant of step 2) to the result of step 1) for emulsifying;

4) a step of adding the ingredient 18 of Table 12 to the resultant of step 3) for thickening; and

5) a step of cooling the resultant of step 4) to 30° C.

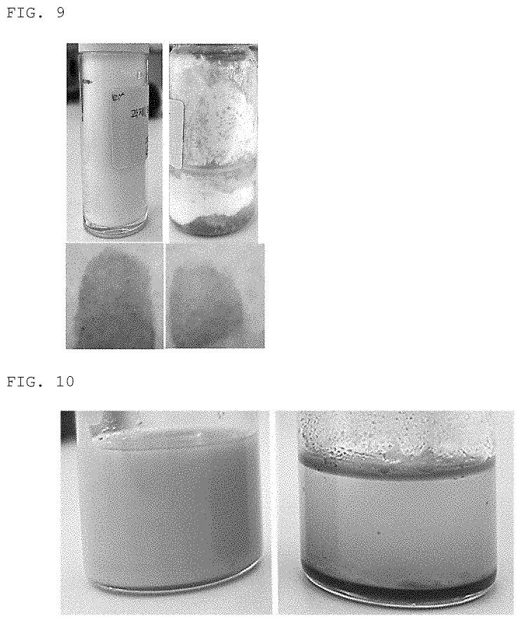

FIG. 9 shows photographs showing the appearances (top) of the formulations of Comparative Example 11 (right photo) and Example 18 (left photo) and the results after each composition was applied to the skin and washed with water. As shown in FIG. 9, in Comparative Example 11 (right photo), as both water and fatty acid were comprised in the formulation, a weak tinting effect was temporarily displayed immediately after preparation, but the formulation was immediately separated. This is due to the disturbance of the emulsion system from simple mixing, rather than the pigment being comprised as a pigment base in a stable state as the pigment is modified to dye. On the other hand, in Example 18 (left photo), it can be seen that not only the tinting was performed effectively, but the formulation was also stable.

TABLE 13
Oil dispersion formulation (anhydrous formulation)

| | | Example | Comparative |
|---|---|---|---|
| | Ingredient name | 19 | Example 12 |
| 1 | polybutene | 32.40 | 32.40 |
| 2 | polyglyceryl-10 decaiisostearate | 20.00 | 20.00 |
| 3 | hydrogenated polyisobutene | 9.00 | 9.00 |
| 4 | diisostearyl malate | 2.60 | 2.60 |
| 5 | tridecyl trimellitate | 13.00 | 13.00 |
| 6 | silica dimethicone silylate | 3.00 | 3.00 |
| 7 | colorant composition of Example 1 | 20.00 | — |
| 8 | stearic acid | — | 1.00 |
| 9 | octyldodecanol | — | 3.00 |
| 10 | polyglyceryl-2 triisostearate | — | 0.70 |
| 11 | tonor pigment(R#6 Na salt(CI 15850)) | — | 0.30 |
| 12 | D.I water | — | To 100 |
| 13 | aminomethyl propanediol | — | 0.20 |

(% by weight)

The composition of oil dispersion formulation of Example 19 was prepared by the following preparation method.

1) a step of adding the ingredients 1 to 6 of Table 13 in a container, and dispersing at 50° C.;

2) a step of adding the ingredient 7 in the container and dispersing it; and

3) a step of cooling and degassing the resultant of step 2) to 30° C.

The composition of oil dispersion formulation of Example 12 was prepared by the following preparation method.

1) a step of adding the ingredients 1 to 6, 8 to 10, 12 and 13 of Table 13 in a container, and dispersing at 50° C.;

2) a step of adding the ingredient 11 in the container and dispersing it; and

3) a step of cooling the resultant of step 2) to 30° C.

As a result, as shown in FIG. 10, in Comparative Example 12 (right photo), the water and oil ingredients repel each other, so the formulation was not mixed and immediately separated. That is, a water layer was formed on the lower layer, and the toner pigment dispersed in the oil sank toward the boundary of the water depending on the conditions, so it was not possible to check whether the tinting was occurred. On the other hand, in Example 19, since the skeleton of the colorant composition itself was stable, the formulation was maintained stably even when it was comprised in the anhydrous formulation. This means that by using the colorant composition according to an embodiment of the present disclosure, it is possible to improve the kind of colorant that can be used, which has been very limited, due to the composition characteristics of an anhydrous formulation such as an oil dispersion formulation.

TABLE 14
Water dispersion formulation

| | | Example | Comparative |
|---|---|---|---|
| | Ingredient name | 20 | Example 13 |
| 1 | D.I water | To 100 | To 100 |
| 2 | glycerine | 16 | 16 |
| 3 | glyceryl polymethacrylate | 25 | 25 |
| 4 | propanediol | 7 | 7 |
| 5 | trehalose | 1 | 1 |
| 6 | tromethamine | 0.3 | 0.3 |
| 7 | 1,2-hexanediol | 1 | 1 |
| 8 | pentylene glycol | 1 | 1 |
| 9 | stearic acid | — | 1.00 |
| 10 | octyldodecnol | — | 3.00 |
| 11 | polyglyceryl-2 triisostearate | — | 0.70 |
| 12 | toner pigment(R#6 Na salt(CI 15850)) | — | 0.30 |
| 13 | colorant composition of Example 1 | 20 | — |
| 14 | ammonium acryloyldimethyltaurate/VP | 0.5 | 0.50 |
| | copolymer | | |

(% by weight)

The composition of water dispersion formulation of Example 20 was prepared by the following preparation method.

1) a step of adding the ingredients 1-8 of Table 14 in a container and dispersing at room temperature;

2) a step of adding the ingredient 13 in the container and dispersing it;

3) a step of adding the ingredient 14 in the container for thickening; and

4) a step of degassing.

The composition of water dispersion formulation of Example 13 was prepared by the following preparation method.

1) a step of adding the ingredients 1-11 of Table 14 in a container, heating to 60° C., and dispersing;

2) a step of adding the ingredient 12 in the container and dispersing it;

3) a step of adding the ingredient 14 to the container for thickening; and

4) a step of cooling the resultant of step 3) to 30° C. and degassing.

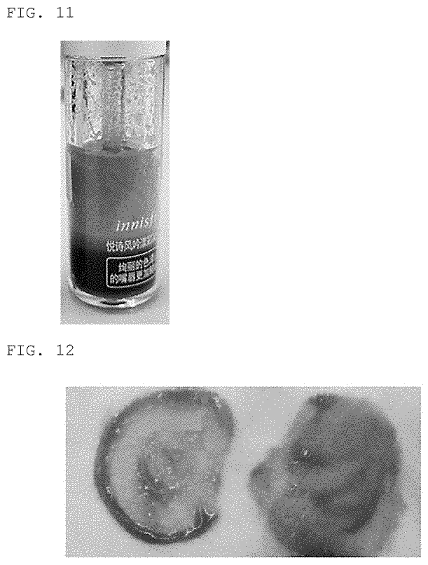

Comparative Example 13 contained the fatty acid and the neutralizing agent, and compared to the oil dispersion (anhydrous) formulation of Table 13, it seemed that formulation was stable and tinting could be occurred at the beginning. However, as a result, as shown in FIG. 11, after 4 months, the insoluble toner pigment directly introduced was precipitated and the formulation was separated. On the other hand, in Example 20, the formulation was prepared transparently and stably. In addition, in Example 12, the actual amount of toner pigment contained in the colorant composition of Example 1 immediately after application was 0.3% by weight relative to the total weight of the composition of Example 20, so the appearance color was weak (the left photo in FIG. 12). However, as the formulation permeated the skin over time, the color was shown due to the tinting (right photo in FIG. 12).

The present disclosure may provide the following embodiments as an example.

A first embodiment provides a colorant composition comprising a saponification reactant, wherein the saponification reactant is the saponification reactant of a toner pigment, a fatty acid and a neutralizing agent.

A second embodiment provides the composition according to the first embodiment, wherein the toner pigment is a water-soluble organic pigment to which a metal salt is bound.

A third embodiment provides the composition according to the first or second embodiment, wherein the fatty acid is the fatty acid having 12 to 22 carbon atoms.

A fourth embodiment provides the composition according to any one of the first to third embodiments, wherein the fatty acid comprises one or more selected from the group consisting of lauric acid, myristic acid, palmitic acid, stearic acid and behenic acid.

A fifth embodiment provides the composition according to any one of the first to fourth embodiments, wherein the neutralizing agent is an amine-based basic material.

A sixth embodiment provides the composition according to any one of the first to fifth embodiments, wherein the neutralizing agent comprises one or more selected from the group consisting of amino methyl propanol, amino methyl propandiol, tromethamine, and triethanolamine.

A seventh embodiment provides the composition according to any one of the first to sixth embodiments, wherein a weight ratio of the fatty acid and the neutralizing agent comprised in the composition is 1:0.1 to 4.

An eighth embodiment provides the composition according to any one of the first to seventh embodiments, wherein the toner pigment comprises one or more selected from the group consisting of a disodium salt of 4-(2-sulfo-p-tolylazo)-3-hydroxy-2-naphthoic acid, a calcium salt of 4-(2-sulfo-p-tolylazo)-3-hydroxy-2-naphthoic acid, and a calcium salt of 4-(1-sulfo-2-naphthylazo)-3-hydroxy-2-naphthoic acid.

A ninth embodiment provides the composition according to any one of the first to eighth embodiments, wherein a weight ratio of the toner pigment, the fatty acid and the neutralizing agent is 0.1-1.5:5:0.5-20.

A tenth embodiment provides the composition according to any one of the first to ninth embodiments, wherein when the composition is applied to a skin, the pigment is tinted to the skin.

An eleventh embodiment provides the composition according to any one of the first to tenth embodiments, wherein a pH of the composition is 7 to 9.5.

A twelfth embodiment provides a cosmetic composition comprising the colorant composition according to any one of the first to eleventh embodiments.

A thirteenth embodiment provides a method for manufacturing the colorant composition according to any one of the first to twelfth embodiments, comprising the step of performing a saponification reaction by adding the neutralizing agent to the fatty acid and the toner pigment.

A fourteenth embodiment provides the method according to the thirteenth embodiment, wherein the step of performing the saponification reaction comprises the steps of dispersing the toner pigment in an oil phase in which the fatty acid is dissolved; and adding a water phase comprising the neutralizing agent to the oil phase to perform the saponification reaction.

A fifteenth embodiment provides the method according to the thirteenth or fourteenth embodiment, wherein the fatty acid is the fatty acid having 12 to 22 carbon atoms.

A sixteenth embodiment provides the method according to any one of the thirteenth to fifteenth embodiments, wherein the fatty acid comprises one or more selected from the group consisting of lauric acid, myristic acid, palmitic acid, stearic acid and behenic acid.

A seventeenth embodiment provides the method according to any one of the thirteenth to sixteenth embodiments, wherein the neutralizing agent is an amine-based basic material.

An eighteenth embodiment provides the method according to any one of the thirteenth to seventeenth embodiments, wherein the neutralizing agent comprises one or more selected from the group consisting of amino methyl propanol, amino methyl propandiol, tromethamine, and triethanolamine.

A nineteenth embodiment provides the method according to any one of the thirteenth to sixteenth embodiments, wherein a weight ratio of the fatty acid and the neutralizing agent is 1:0.1 to 4.

A twentieth embodiment provides the method according to any one of the thirteenth to nineteenth embodiments, wherein the toner pigment comprises one or more selected from the group consisting of a disodium salt of 4-(2-sulfo-p-tolylazo)-3-hydroxy-2-naphthoic acid, a calcium salt of 4-(2-sulfo-p-tolylazo)-3-hydroxy-2-naphthoic acid, and a calcium salt of 4-(1-sulfo-2-naphthylazo)-3-hydroxy-2-naphthoic acid.

What is claimed is:

1. A colorant composition comprising a saponification reactant, wherein the saponification reactant is the saponification reactant of a toner pigment, a fatty acid and a neutralizing agent.

2. The colorant composition according to claim 1, wherein the toner pigment is a water-soluble organic pigment to which a metal salt is bound.

3. The colorant composition according to claim 1, wherein the fatty acid is the fatty acid having 12 to 22 carbon atoms.

4. The colorant composition according to claim 1, wherein the fatty acid comprises one or more selected from the group consisting of lauric acid, myristic acid, palmitic acid, stearic acid and behenic acid.

5. The colorant composition according to claim 1, wherein the neutralizing agent is an amine-based basic material.

6. The colorant composition according to claim 1, wherein the neutralizing agent comprises one or more selected from the group consisting of amino methyl propanol, amino methyl propandiol, tromethamine, and triethanolamine.

7. The colorant composition according to claim 1, wherein a weight ratio of the fatty acid and the neutralizing agent comprised in the composition is 1:0.1 to 4.

8. The colorant composition according to claim 1, wherein the toner pigment comprises one or more selected from the group consisting of a disodium salt of 4-(2-sulfo-p-tolylazo)-3-hydroxy-2-naphthoic acid, a calcium salt of 4-(2-sulfo-p-tolylazo)-3-hydroxy-2-naphthoic acid, and a calcium salt of 4-(1-sulfo-2-naphthylazo)-3-hydroxy-2-naphthoic acid.

9. The colorant composition according to claim 1, wherein a weight ratio of the toner pigment, the fatty acid and the neutralizing agent is 0.1-1.5:5:0.5-20.

10. The colorant composition according to claim 1, wherein when the composition is applied to a skin, the pigment is tinted to the skin.

11. The colorant composition according to claim 1, wherein a pH of the composition is 7 to 9.5.

12. A cosmetic composition comprising the colorant composition of claim 1.

13. A method for manufacturing the colorant composition according to claim 1, comprising the step of performing saponification reaction by adding the neutralizing agent to the fatty acid and the toner pigment.

14. The method according to claim 13, wherein the step of performing the saponification reaction comprises the steps of:
dispersing the toner pigment in an oil phase in which the fatty acid is dissolved; and
adding a water phase comprising the neutralizing agent to the oil phase to perform the saponification reaction.

15. The method according to claim 13, wherein the fatty acid is the fatty acid having 12 to 22 carbon atoms.

16. The method according to claim 13, wherein the fatty acid comprises one or more selected from the group consisting of lauric acid, myristic acid, palmitic acid, stearic acid and behenic acid.

17. The method according to claim 13, wherein the neutralizing agent is an amine-based basic material.

18. The method according to claim 13, wherein the neutralizing agent comprises one or more selected from the group consisting of amino methyl propanol, amino methyl propandiol, tromethamine, and triethanolamine.

19. The method according to claim 13, wherein a weight ratio of the fatty acid and the neutralizing agent is 1:0.1 to 4.

20. The method according to claim 13, wherein the toner pigment comprises one or more selected from the group consisting of a disodium salt of 4-(2-sulfo-p-tolylazo)-3-hydroxy-2-naphthoic acid, a calcium salt of 4-(2-sulfo-p-tolylazo)-3-hydroxy-2-naphthoic acid, and a calcium salt of 4-(1-sulfo-2-naphthylazo)-3-hydroxy-2-naphthoic acid.
